(19) United States
(12) Patent Application Publication
(10) Pub. No.: US 2014/0363330 A1
(43) Pub. Date: Dec. 11, 2014

(54) METHOD OF ENHANCING SOFT TISSUE INTEGRATION AND SEAL AROUND PROSTHETIC DEVICES

(71) Applicant: The Regents of the University of California, Oakland, CA (US)

(72) Inventor: Takahiro Ogawa, Torrance, CA (US)

(21) Appl. No.: 14/373,331

(22) PCT Filed: Jan. 14, 2013
(86) PCT No.: PCT/US2013/021437

§ 371 Date: Jul. 18, 2014

Related U.S. Application Data
(60) Provisional application No. 61/588,582, filed on Jan. 19, 2012.

(51) Int. Cl. A61L 27/06; A61B 17/80; A61L 27/16; A61L 27/50; A61L 27/10; A61L 27/12; A61L 27/02; A61L 27/26; A61F 2/02; A61L 27/18

(52) U.S. Cl. CPC A61L 27/06; A61F 2/02; A61B 17/80; A61L 27/16; A61L 27/18; A61L 27/10; A61L 27/12; A61L 27/025; A61L 27/26; A61L 27/50; A61L 2400/18
USPC 420/417; 526/72; 528/271; 526/329.7; 523/113; 523/116; 525/227; 525/191; 423/305; 501/1; 501/11; 106/286.4; 250/492.1

(57) ABSTRACT

Provided herein are methods of enhancing soft tissue integration with and seal around prosthetic devices.

### CROSS-REFERENCE TO RELATED APPLICATIONS

This invention claims the benefit of U.S. provisional application No. 61/588,582, filed on Jan. 19, 2012, the teaching of which is incorporated herein in its entirety by reference.

### BACKGROUND OF THE INVENTION

1. Field of the Invention

This invention generally relates to methods of enhancing soft tissue integration with and seal around prosthetic devices.

2. Description of the Background

Dental crowns are placed on remaining structure of teeth after tooth decay that destructs a significant part of the tooth structure. Dental bridges are also used to restore missing teeth using adjacent teeth as anchors. Because these prosthodontic devices are in direct contact with periodontal mucosal tissue (gum tissue), biological behavior and response of the tissue to the marginal area of the devices directly affect the subsequent periodontal health and prognosis of the teeth[1-3]. Periodontal inflammation, called gingivitis or periodontitis (gum disease), involves gum bleeding, swelling, resorption of alveolar bone supporting the teeth, the recession of gum and bone, and loosening of the teeth and eventually becomes a primary reason for tooth loss[4, 5].

Restorative treatment of missing teeth via dental implants has a considerable effect on oral health: masticatory function[6,7], speech[8] and quality of life[9] are improved as compared to conventional removable denture prostheses. In the U.S, 10% of the adults and the one-third of adults aged >65 years are fully edentulous [10,11]. Despite its increasing need in an aging society, dental implant therapy has been employed in only 2% of the potential patients[12]. Limitation and current challenge of dental implant treatment is a destructive change of surrounding tissue (gum and bone) around implants. Measures to maintain short and long term health of surrounding gum and bone tissues are urgently desired[13-17]. A primary reason for implant failure is post-implantation inflammation, referred to as peri-implantitis[18-21]. Such inflammation causes the infection and destructive cascade around bone and gum tissues around the implants, leading to a loosening and failure of implants. A top portion of implant fixtures and related devices such as healing abutments and connecting abutments are in direct contact with periodontal soft tissues.

Maxillofacial implants are used for tissue defects caused by injury and cancer in the area, on which prosthetics, such as polymer-made epitheses, obturators and other dentures, are placed via connecting abutments, retention bars, magnets, or other types of attachment devices[22, 23]. These implants as well as connection abutments and devices (such as bars and coping) are trans-mucosa, tans-gum, or trans-skin and subjected to bacterial, chemical contamination and invasion. Therefore, hygiene status and resistance to such unwelcome exogenous stimulation is extremely important for the prognosis of maxillofacial implants and related prostheses [24].

Therefore, technologies to enhance the biological behavior and response of soft tissues hold a key to further improve various prosthetic devices and implants that are used in contact with gum and skin, and trans-gum and -skin. Specifically, measures to establish a barrier and prevent bacterial and chemical invasion to internal biological system through around the prosthetic devices are of extreme desire.

We previously discovered UV treatment-enhanced bone-implant integration. Bone integration is formed by bone cells (osteoblasts alone), while the soft tissue integration is formed by fibroblasts and other types of soft tissue cells, such as epithelial cells, connective tissue cells. Osteoblasts and soft tissue cells are from different origin during the development stage: Osteoblasts are from mesenchymal cells from mesoderm, while epithelial cells stem from ectoderm. Osteoblasts are differentiating cells that changes in their function and behavior during their maturation process, while soft tissue cells are in a mono-character during their life. In fact, osteoblasts and soft tissue cells behave and act very differently. For example, osteoblasts and soft tissue cells respond oppositely on material surfaces [25-28]. In terms of cell adhesion to materials, osteoblasts and fibroblasts respond distinctively and often oppositely [28, 29]. In the process of bone integration around biomaterials, soft tissue formation and bone formation are competing biological events each other and researchers have attempted to develop better biomaterial surfaces to specifically increase osteoblast function and suppress soft tissue cell function[25, 28, 30], which is also an example of different behavior and function between bone cells and soft tissue cells. Therefore, this invention, that demonstrated the soft tissue integration is enhanced on UV treated material surfaces, is of great significance. Also, as described above, therapeutic and physiological roles of bone integration and soft tissue information are completely different.

The embodiments described below address the above identified issues and needs.

### SUMMARY OF THE INVENTION

In one aspect of the present invention, it is provided a prosthetic device, having an enhanced soft tissue integration and seal. The prosthetic device is treated by ultraviolet light (UV) for a period of time of sufficient length prior to implantation of the prosthetic device in a subject so as to impart electrostatics to the surface of the device, wherein the enhanced soft tissue integration and seal is a soft tissue integration with and seal around the prosthetic device that is enhanced by about 10% or above as compared with a device without UV treatment.

In some embodiments of the invention prosthetic device, the soft tissue comprises gingival cells or epithelial cells and/or fibroblast cells.

In some embodiments of the invention prosthetic device, optionally in combination with any or all of the various embodiments disclosed above or below, the prosthetic device is a dental implant.

In some embodiments of the invention prosthetic device, optionally in combination with any or all of the various embodiments disclosed above or below, the prosthetic device is an orthopedic implant.

In some embodiments of the invention prosthetic device, optionally in combination with any or all of the various embodiments disclosed above or below, the prosthetic device is a dental implant selected from the group consisting of dental crowns, bridges, implant fixtures, implant abutment components, attachments, bars, and a superstructure to retain and support prostheses that contact soft tissues.

In some embodiments of the invention prosthetic device, optionally in combination with any or all of the various embodiments disclosed above or below, the prosthetic device is an orthopedic implant selected from the group consisting of femoral stems, knee implants, spine screws, and plates.

In some embodiments of the invention prosthetic device, optionally in combination with any or all of the various embodiments disclosed above or below, the prosthetic device comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the invention prosthetic device, optionally in combination with any or all of the various embodiments disclosed above or below, the prosthetic device is selected from the group consisting of jaw bone prosthetic device, repairing and stabilizing screws, pins, frames, and plates for bone, spinal prosthetic devices, femoral prosthetic devices, neck prosthetic devices, knee prosthetic devices, wrist prosthetic devices, joint prosthetic devices, maxillofacial prosthetic, limb prostheses for conditions resulting from injury and disease, and combinations thereof.

In some embodiments of the invention prosthetic device, optionally in combination with any or all of the various embodiments disclosed above or below, the prosthetic device comprises a polymeric material or a bone cement material. In some embodiments, the bone cement material comprises a material selected from the group consisting of polyacrylates, polyesters, poly(methyl methacrylate) (PMMA) or methyl methacrylate (MMA), bioglass, ceramics, calcium-based materials, calcium phosphate-based materials, and combinations thereof.

In some embodiments of the invention prosthetic device, optionally in combination with any or all of the various embodiments disclosed above or below, the UV light is of an intensity of about 0.05 mW/cm² to about 4.0 mW/cm² of a wave length from about 400 nm to about 100 nm.

In some embodiments of the invention prosthetic device, optionally in combination with any or all of the various embodiments disclosed above or below, the electrostatic properties comprise positive charges ranging from 0.01 nC to 10.00 nC.

In another aspect of the present invention, it is provided a method, comprising treating a prosthetic device with ultraviolet light prior to implantation of the prosthetic device in a subject for a period of time of sufficient length to impart electrostatics to the surface of the device, and wherein the enhanced soft tissue integration and seal is a soft tissue integration with and seal around the prosthetic device that is enhanced by about 10% or above as compared with a device without UV treatment.

In some embodiments of the invention method, optionally in combination with any or all of the various embodiments disclosed above or below, the period of time is about 20 minutes or longer.

In some embodiments of the invention method, optionally in combination with any or all of the various embodiments disclosed above or below, the UV light is has an intensity of about 0.05 mW/cm² to about 4.0 mW/cm² of a wave length from about 400 nm to about 100 nm.

In some embodiments of the invention method, optionally in combination with any or all of the various embodiments disclosed above or below, the electrostatic properties comprise positive charges ranging from 0.01 nC to 10.00 nC.

In some embodiments of the invention method, optionally in combination with any or all of the various embodiments disclosed above or below, the prosthetic device comprises a metallic material.

In some embodiments of the invention method, optionally in combination with any or all of the various embodiments disclosed above or below, the prosthetic device comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the invention method, optionally in combination with any or all of the various embodiments disclosed above or below, the prosthetic device is selected from the group consisting of tooth prosthetic devices, jaw bone prosthetic device, repairing and stabilizing screws, pins, frames, and plates for bone, spinal prosthetic devices, femoral prosthetic devices, neck prosthetic devices, knee prosthetic devices, wrist prosthetic devices, joint prosthetic devices, maxillofacial prosthetic, limb prostheses for conditions resulting from injury and disease, and combinations thereof.

In some embodiments of the invention method, optionally in combination with any or all of the various embodiments disclosed above or below, the prosthetic device comprises a polymeric material or a bone cement material.

In some embodiments of the invention method, optionally in combination with any or all of the various embodiments disclosed above or below, the bone cement material comprises a material selected from the group consisting of polyacrylates, polyesters, poly(methyl methacrylate) (PMMA) or methyl methacrylate (MMA), bioglass, ceramics, calcium-based materials, calcium phosphate-based materials, and combinations thereof.

In another aspect of the present invention, it is provided a method of treating a medical condition in a subject, comprising implanting in the subject a prosthetic device in need thereof, wherein the prosthetic device is as the various embodiments of invention prosthetic device disclosed above or below. In some embodiments, the medical condition is a dental condition. In some embodiments, the medical condition is a bone-related condition.

### BRIEF DESCRIPTION OF THE DRAWINGS

FIG. 1 shows enhanced adhesion of gum tissues on UV-treated titanium metal surface.

FIG. 2 shows enhanced adhesion of skin tissues on UV-treated titanium metal surface.

FIG. 3 shows enhanced adhesion of gum tissues on UV-treated gold alloy metal surface.

FIG. 4 shows enhanced adhesion of gingival cells on UV-treated titanium metal surface.

FIG. 5 shows enhanced adhesion of fibroblast cells on UV-treated titanium metal surface.

FIG. 6 shows XPS measurements showing that UV-treated titanium surfaces have a lower percentage of atomic carbon (less than 20%) than untreated titanium surfaces (above 45%).

FIG. 7 demonstrates the change of surface electric charge of UV treated metals.

### DETAILED DESCRIPTION

In one aspect of the present invention, it is provided a prosthetic device, having an enhanced soft tissue integration and seal. The prosthetic device is treated by ultraviolet light prior to implantation of the prosthetic device in a subject for a period of time of sufficient length so as to impart electrostatics to the surface of the device, wherein the enhanced soft tissue integration and seal is a soft tissue integration with and seal around the prosthetic device that is enhanced by about 10% or above as compared with a device without UV treatment.

In some embodiments of the invention prosthetic device, the soft tissue comprises gingival cells or epithelial cells and/or fibroblast cells.

In some embodiments of the invention prosthetic device, optionally in combination with any or all of the various embodiments disclosed above or below, the prosthetic device is a dental implant.

In some embodiments of the invention prosthetic device, optionally in combination with any or all of the various embodiments disclosed above or below, the prosthetic device is an orthopedic implant.

In some embodiments of the invention prosthetic device, optionally in combination with any or all of the various embodiments disclosed above or below, the prosthetic device is a dental implant selected from the group consisting of dental crowns, bridges, implant fixtures, implant abutment components, attachments, bars, and a superstructure to retain and support prostheses that contact soft tissues.

In some embodiments of the invention prosthetic device, optionally in combination with any or all of the various embodiments disclosed above or below, the prosthetic device is an orthopedic implant selected from the group consisting of femoral stems, knee implants, spine screws, and plates.

In some embodiments of the invention prosthetic device, optionally in combination with any or all of the various embodiments disclosed above or below, the prosthetic device comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the invention prosthetic device, optionally in combination with any or all of the various embodiments disclosed above or below, the prosthetic device is selected from the group consisting of jaw bone prosthetic device, repairing and stabilizing screws, pins, frames, and plates for bone, spinal prosthetic devices, femoral prosthetic devices, neck prosthetic devices, knee prosthetic devices, wrist prosthetic devices, joint prosthetic devices, maxillofacial prosthetic, limb prostheses for conditions resulting from injury and disease, and combinations thereof.

In some embodiments of the invention prosthetic device, optionally in combination with any or all of the various embodiments disclosed above or below, the prosthetic device comprises a polymeric material or a bone cement material. In some embodiments, the bone cement material comprises a material selected from the group consisting of polyacrylates, polyesters, poly(methyl methacrylate) (PMMA) or methyl methacrylate (MMA), bioglass, ceramics, calcium-based materials, calcium phosphate-based materials, and combinations thereof.

In some embodiments of the invention prosthetic device, optionally in combination with any or all of the various embodiments disclosed above or below, the UV light is has an intensity of about 0.05 mW/cm² to about 4.0 mW/cm² of a wave length from about 400 nm to about 100 nm.

In some embodiments of the invention prosthetic device, optionally in combination with any or all of the various embodiments disclosed above or below, the electrostatic properties comprise positive charges ranging from 0.01 nC to 10.00 nC.

In another aspect of the present invention, it is provided a method, comprising treating a prosthetic device with ultraviolet light prior to implantation of the prosthetic device in a subject for a period of time of sufficient length to impart electrostatics to the surface of the device, and wherein the enhanced soft tissue integration and seal is a soft tissue integration with and seal around the prosthetic device that is enhanced by about 10% or above as compared with a device without UV treatment.

In some embodiments of the invention method, optionally in combination with any or all of the various embodiments disclosed above or below, the period of time is about 20 minutes or longer. The time of UV treatment is conversely related to the UV intensity. Generally speaking, treatment of the prosthetic device disclosed herein using UV having an higher intensity would require a shorter time of UV treatment, and vice versa.

In some embodiments of the invention method, optionally in combination with any or all of the various embodiments disclosed above or below, the UV light is of an intensity of about 0.05 mW/cm² to about 4.0 mW/cm² of a wave length from about 400 nm to about 100 nm, e.g., 0.5 mW/cm (λ=360±20 nm) or 1.5 mW/cm² (λ=250±20 nm). In some embodiments, stronger (higher intensity) or weaker (lower intensity) UV light can be used. For example, the UV light can have an intensity below 0.5 mW/cm², such as about 0.05 mW/cm² (λ.=360±20 nm), about 0.1 mW/cm² (λ=360±20 nm), about 0.2 mW/cm² (λ=360±20 nm), about 0.3 mW/cm² (λ=360±20 nm), or about 0.4 mW/cm² (λ=360±20 nm). In some embodiments, the UV light can have an intensity above 1.5 mW/cm², such as about 2.0 mW/cm² (λ=250±20 nm), about 2.5 mW/cm² (λ=250±20 nm), about 3.0 mW/cm² (λ=250±20 nm), about 3.5 mW/cm² (λ=250±20 nm), about 4.0 mW/cm² (λ=250±20 nm) or above, provided that the intensity is below that of a laser.

In some embodiments of the invention method, optionally in combination with any or all of the various embodiments disclosed above or below, the electrostatic properties comprise positive charges ranging from 0.01 nC to 10.00 nC.

Note, UV lights having an intensity described herein can have a wave length that is common for a UV light device, such as λ=360±20 nm, λ=250±20 nm, or another wave length within the UV range from 400 nm to 100 nm, such as UVA, UVB, or UVC, which are described further below.

In some embodiments of the invention method, optionally in combination with any or all of the various embodiments disclosed above or below, the prosthetic device comprises a metallic material.

In some embodiments of the invention method, optionally in combination with any or all of the various embodiments disclosed above or below, the prosthetic device comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

In some embodiments of the invention method, optionally in combination with any or all of the various embodiments disclosed above or below, the prosthetic device is selected from the group consisting of tooth prosthetic devices, jaw bone prosthetic device, repairing and stabilizing screws, pins, frames, and plates for bone, spinal prosthetic devices, femoral prosthetic devices, neck prosthetic devices, knee prosthetic devices, wrist prosthetic devices, joint prosthetic devices, maxillofacial prosthetic, limb prostheses for conditions resulting from injury and disease, and combinations thereof.

In some embodiments of the invention method, optionally in combination with any or all of the various embodiments disclosed above or below, the prosthetic device comprises a polymeric material or a bone cement material.

In some embodiments of the invention method, optionally in combination with any or all of the various embodiments disclosed above or below, the bone cement material comprises a material selected from the group consisting of polyacrylates, polyesters, poly(methyl methacrylate) (PMMA) or methyl methacrylate (MMA), bioglass, ceramics, calcium-based materials, calcium phosphate-based materials, and combinations thereof.

In another aspect of the present invention, it is provided a method of treating a medical condition in a subject, comprising implanting in the subject a prosthetic device in need thereof, wherein the prosthetic device is as the various embodiments of invention prosthetic device disclosed above or below. In some embodiments, the medical condition is a dental condition. In some embodiments, the medical condition is a bone-related condition.

As used herein, Ultraviolet (UV) light is electromagnetic radiation with a wavelength shorter than that of visible light, but longer than X-rays, that is, in the range 10 nm to 400 nm, corresponding to photon energies from 3 eV to 124 eV. As used herein, the term treating with an ultraviolet light “UV” can be used interchangeably with the term “light activation,” “light radiation,” “light irradiation,” “UV light activation,” “UV light radiation,” or “UV light irradiation.” UV lights can be divided into UVA (400 nm to 315 nm), UVB (315 nm to 280 nm), and UVC (280 nm to 100 nm). Different wave length of UV, such as UVA, UVB, and UVC, imparts properties to UV lights that can be very different. For example, UVC is germicidal while UVA may be less effective as germicide.

As used herein, the term “UV” or “UV light” shall not encompass a UV laser or UV laser beam. Such UV light does not encompass any UV beam obtained through optical amplification such as those fall within the definition of laser as described in Gould, R. Gordon (1959). “The LASER, Light Amplification by Stimulated Emission of Radiation”. In Franken, P. A. and Sands, R. H. (Eds.). The Ann Arbor Conference on Optical Pumping, the University of Michigan, 15 June through 18 Jun. 1959. p. 128.

Examples of UV light used herein have the ca. 0.5 mW/cm² (λ=360±20 nm) and 1.5 mW/cm² (λ=250±20 nm).

As used herein, the term “carbon content” refers to any contamination in air containing carbon that is not carbon dioxide. Such contamination can be any organic species, carbon particles, or an inorganic compound in the air that contains carbon.

As used herein, the term “tissue integration capability” refers to the ability of a prosthetic device to be integrated into the tissue of a biological body. The tissue integration capability of a prosthetic device can be generally measured by several factors, one of which is wettability of the prosthetic device surface, which reflects the hydrophilicity/oleophilicty (hydrophobicity), or hemophilicity of a prosthetic device surface. Hydrophilicity and oleophilicity are relative terms and can be measured by, e.g., water contact angle (Oshida Y, et al., J Mater Science 3:306-312 (1992)), and area of water spread (Gifu-kosen on line text, http://www.gifu-nct.ac.jp/elec/tokoro/fft/contact-angle.html). For purposes of the present invention, the hydrophilicity/oleophilicity can be measured by contact angle or area of water spread of a prosthetic device surface described herein relative to the ones of the control prosthetic device surfaces. Relative to the prosthetic device surfaces not treated with the process described herein, a prosthetic device treated with the process described herein has a substantially lower contact angle or a substantially higher area of water spread.

As used herein, the term “electrostatic properties” shall mean electric charge on the surface. Such electric charge can be positive or negative. In some embodiments, positive charges can be, for example, charges on a metal atom or metal oxide, for example, Ti(+), Ti(+2), Ti(+3), or Ti(+4) or TiO(+1) or TiO(+2), etc. In some embodiments, such electrostatic properties can be positive charges having a monovalent positivity, which is demonstrated by the fact they can be neutralized by adding monovalent anions. In some embodiments, such electrostatic properties can be positive charges ranging from 0.01 nC to 10.00 nC.

### Prosthetic Devices

The prosthetic devices described herein with enhanced tissue integration capabilities include any prosthetic devices currently available in medicine or to be introduced in the future. The prosthetic devices can be metallic or non-metallic prosthetic devices. Non-metallic prosthetic devices include, for example, ceramic prosthetic devices, calcium phosphate or polymeric prosthetic devices. Useful polymeric prosthetic devices can be any biocompatible prosthetic devices, e.g., bio-degradable polymeric prosthetic devices. Representative ceramic prosthetic devices include, e.g., bioglass and silicon dioxide prosthetic devices. Calcium phosphate prosthetic devices includes, e.g., hydroxyapatite, tricalcium phosphate (TCP). Exemplary polymeric prosthetic devices include, e.g., poly-lactic-co-glycolic acid (PLGA), polyacrylate such as polymethacrylates and polyacrylates, and poly-lactic acid (PLA) prosthetic devices. In some embodiments, the prosthetic device described herein can specifically exclude any of the aforementioned materials.

In some embodiments, the prosthetic device comprises a metallic prosthetic device and a bone-cement material. The bone cement material can be any bone cement material known in the art. Some representative bone cement materials include, but are not limited to, polyacrylate or polymethacrylate based materials such as poly(methyl methacrylate)(PMMA)/methyl methacrylate (MMA), polyester based materials such as PLA or PLGA, bioglass, ceramics, calcium phosphate-based materials, calcium-based materials, and combinations thereof. In some embodiments, the prosthetic device can include any polymer described below. In some embodiments, the prosthetic device described herein can specifically exclude any of the aforementioned materials.

The metallic prosthetic devices described herein include titanium prosthetic devices and non-titanium prosthetic devices. Titanium prosthetic devices include tooth or bone replacements made of titanium or an alloy that includes titanium. Titanium bone replacements include, e.g., knee joint and hip joint prostheses, femoral neck replacement, spine replacement and repair, neck bone replacement and repair, jaw bone repair, fixation and augmentation, transplanted bone fixation, and other limb prostheses. None-titanium metallic prosthetic devices include tooth or bone prosthetic devices made of gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, e.g., stainless steel, or combinations thereof. Some examples of alloys are titanium-nickel allows such as nitanol, chromium-cobalt alloys, stainless steel, or combinations thereof. In some embodiments, the metallic prosthetic device can specifically exclude any of the aforementioned metals.

The prosthetic device described herein can be porous or non-porous prosthetic devices. Porous prosthetic devices can impart better tissue integration while non-porous prosthetic devices can impart better mechanical strength.

The prosthetic devices can be metallic prosthetic devices or non-metallic prosthetic devices. In some embodiments, the prosthetic devices are metallic prosthetic devices such as titanium prosthetic devices, e.g., titanium prosthetic devices for replacing missing teeth (dental prosthetic devices) or fixing diseased, fractured or transplanted bone. Other exemplary metallic prosthetic devices include, but are not limited to, titanium alloy prosthetic devices, chromium-cobalt alloy prosthetic devices, platinum and platinum alloy prosthetic devices, nickel and nickel alloy prosthetic devices, stainless steel prosthetic devices, zirconium, chromium-cobalt alloy, gold or gold alloy prosthetic devices, and aluminum or aluminum alloy prosthetic devices.

The prosthetic devices provided herein can be subjected to various established surface treatments to increase surface area or surface roughness for better tissue integration or tissue attachment. Representative surface treatments include, but are not limited to, physical treatments and chemical treatments. Physical treatments include, e.g., machined process, sandblasting process, metallic deposition, non-metallic deposition (e.g., apatite deposition), or combinations thereof. Chemical treatment includes, e.g., etching using a chemical agent such as an acid, base (e.g., alkaline treatment), oxidation (e.g., heating oxidation and anodic oxidation), and combinations thereof. For example, a metallic prosthetic device can form different surface topographies by a machined process or an acid-etching process.

### Polymers

The polymers can be any polymer commonly used in the medical device industry. The polymers can be biocompatible or non-biocompatible. In some embodiments, the polymer can be poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), polypropylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), polypropylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), molecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof. Some examples of elastin protein mimetics include (LGGVG)ₙ, (VPGVG)ₙ, Val-Pro-Gly-Val-Gly, or synthetic biomimetic poly(L-glytanmate)-b-poly(2-acryloyloxyethyllactoside)-b-poly(1-glutamate) triblock copolymer.

In some embodiments, the polymer can be poly(ethylene-co-vinyl alcohol), poly(methoxyethyl methacrylate), poly(dihydroxylpropyl methacrylate), polymethacrylamide, aliphatic polyurethane, aromatic polyurethane, nitrocellulose, poly(ester amide benzyl), co-poly-{[N,N′-sebacoyl-bis-(L-leucine)-1,6-hexylene diester]_{0.75}-[N,N′-sebacoyl-L-lysine benzyl ester]_{0.25}} (PEA-Bz), co-poly-{[N,N′-sebacoyl-bis-(L-leucine)-1,6-hexylene diester]0.75-[N,N′-sebacoyl-L-lysine-4-amino-TEMPO amide]_{0.25}} (PEA-TEMPO), aliphatic polyester, aromatic polyester, fluorinated polymers such as poly(vinylidene fluoride-co-hexafluoropropylene), poly(vinylidene fluoride) (PVDF), and Teflon™ (polytetrafluoroethylene), a biopolymer such as elastin mimetic protein polymer, star or hyper-branched SIBS (styrene-block-isobutylene-block-styrene), or combinations thereof. In some embodiments, where the polymer is a copolymer, it can be a block copolymer that can be, e.g., di-, tri-, tetra-, or oligo-block copolymers or a random copolymer. In some embodiments, the polymer can also be branched polymers such as star polymers.

In some embodiments, a UV-transmitting material having the features described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Medical Use

The prosthetic devices provided herein can be used for treating, preventing, ameliorating, correcting, or reducing the symptoms of a medical condition by implanting the prosthetic devices in a mammalian subject. The mammalian subject can be a human being or a veterinary animal such as a dog, a cat, a horse, a cow, a bull, or a monkey.

Representative medical conditions that can be treated or prevented using the prosthetic devices provided herein include, but are not limited to, missing teeth or bone related medical conditions such as femoral neck fracture, missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a disorder or body condition such as, e.g., cancer, injury, systemic metabolism, infection or aging, and combinations thereof.

In some embodiments, the prosthetic devices provided herein can be used to treat, prevent, ameliorate, or reduce symptoms of a medical condition such as missing teeth, a need for orthodontic anchorage or bone related medical conditions such as femoral neck fracture, neck bone fracture, wrist fracture, spine fracture/disorder or spinal disk displacement, fracture or degenerative changes of joints such as knee joint arthritis, bone and other tissue defect or recession caused by a body condition or disorder such as cancer, injury, systemic metabolism, infection and aging, limb amputation resulting from injuries and diseases, and combinations thereof.

### EXAMPLES

The following examples illustrate, and shall not be construed to limit, the embodiments of the present invention.

### Summary

Here, we have discovered that UV light treatment of prosthetic materials significantly enhance the adhesion and retention of the soft tissues (gum and skin tissues) and soft-tissue cells, leading to a remarkably greater degree of soft tissue integration. Because the degree of soft tissue adhesion/integration determines the degree of soft tissue seal from the surrounding environments and protects the internal biological cells, tissues and structures, it can be an efficient and promising measure to maintain short- and long-term health of biological tissues around the prostheses and related devices. The surfaces of the UV-treated materials show a significantly reduced level of surface carbon and positive electric charge. The UV-mediated enhancement of soft tissue integration is expected to be applied to any types of prosthetic devices and components that are required for soft tissue biocompatibility and integration, including but not limited to dental crowns, bridges, implant fixtures, implant abutment components, attachments, bars, any types of superstructures to retain and support prostheses that contact soft tissues, and orthopedic implants such as femoral stems, knee implants, spine screws, and plates.

### Materials and Methods

### Samples

Disks (20 mm in diameter and 1.0 mm in thickness) made of commercially pure titanium (Grade 2) and gold-alloy were used. UV treatment was performed for 20 min using UV light; intensity, ca. 0.5 mW/cm² (λ=360±20 nm) and 1.5 mW/cm² (λ=250±20 nm). The chemical composition on titanium surfaces were evaluated by electron spectroscopy for chemical analysis (ESCA). ESCA was performed using an X-ray photoelectron spectroscopy (XPS) (ESCA3200, Shimadzu, Tokyo, Japan) under high vacuum conditions (6×10⁻⁷ Pa).

### Electrostatic Treatment of Material Surfaces

To identify the role of surface electrostatic status of UV-treated surfaces in determining cell adhesion, cell adhesion was examined on UV-treated titanium surface with an additional electrostatic treatment. Titanium disks after UV treatment were incubated for 1 h at room temperature in 1 ml of 0.1 M NaCl. The disks were then washed twice with ddH₂O and left to completely dry at room temperature for 1 h before seeding cells.

### Cell and Tissue Culture

Gingival cells isolated from upper jaw palatal tissues of 8-week-old male Sprague-Dawley rats and NIH3T3 fibroblasts were placed into Dulbecco's Modified Eagle Medium (Gibco BRL, Grand Island, N.Y.), supplemented with 10% Fetal Bovine Serum and antibiotic-antimycotic solution containing 10000 units/ml penicillin G sodium, 10000 mg/ml streptomycin sulfate and 25 mg/ml amphotericin B. Cells were incubated in a humidified atmosphere of 95% air, 5% CO₂ at 37° C. At 80% confluency, the cells were detached using 0.25% Trypsin-1 mM EDTA-4Na and seeded onto metal disks. Gingival tissues (2 mm×2 mm) and skin tissues (2 mm×2 mm) were isolated, respectively, from rat palatal gingiva and dorsal skin and cultured in the same way of cells.

### Cell and Tissue Adhesion Assay

The adhesive strength of cells attached to material surfaces was evaluated by the percentage of detached cells after mechanical detachment. Cells incubated on disks for 24 h were rinsed once with PBS to remove non-adherent cells, and then detached from the surfaces by agitating (frequency, 35 Hz; 3 mm, amplitude). The detached and remaining cells were quantified with WST-1 assay. Tissues adhesion assay was performed in a similar way. The tissues were adhered to disks for 2 or 3 days before detachment.

### Results

### Enhanced Adhesion of Gum Tissues on UV-Treated Metal

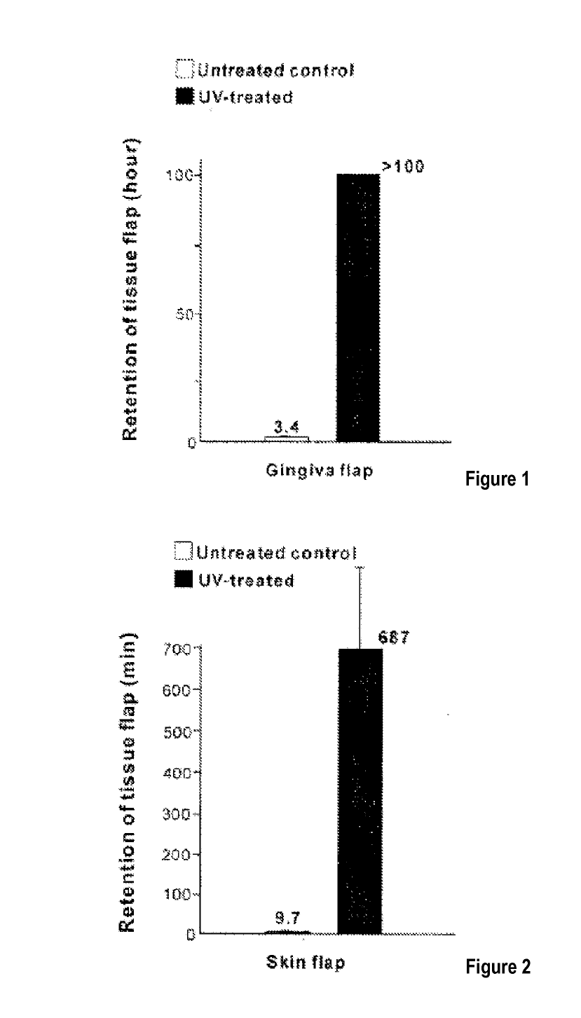

Tissue flaps (2 mm×2 mm) of gum (gingival mucosa) isolated from rat upper jaw were placed on titanium disks with and without UV treatment. The gum tissues were incubated in the culture medium for 3 days to obtain the initial attachment to titanium disks. Then, the culture dish was shaken on an agitating device to detach from titanium disks. The gum tissues were retained on UV-treated titanium disks until 100 h without detachment. The measurement was discontinued at 100 h and there is a possibility the tissues remained for even longer time. The gum tissues on untreated titanium disks were detached within 3.5 hours (FIG. 1).

### Enhanced Adhesion of Skin Tissues on UV-Treated Metal

The 2 mm×2 mm skin tissues isolated from rat dorsal skin was placed on titanium disks with and without UV treatment. The skin tissues were incubated in the culture medium for 2 days to obtain the initial attachment to titanium disks. Then, the culture dish was shaken on an agitating device to detach from titanium disks. The skin tissues were retained on UV treated titanium disks for longer than 650 min without detachment, while the skin tissues on untreated titanium disks were detached within 10 min (FIG. 2).

### Enhanced Adhesion of Gum Tissues on UV-Treated Other Metal

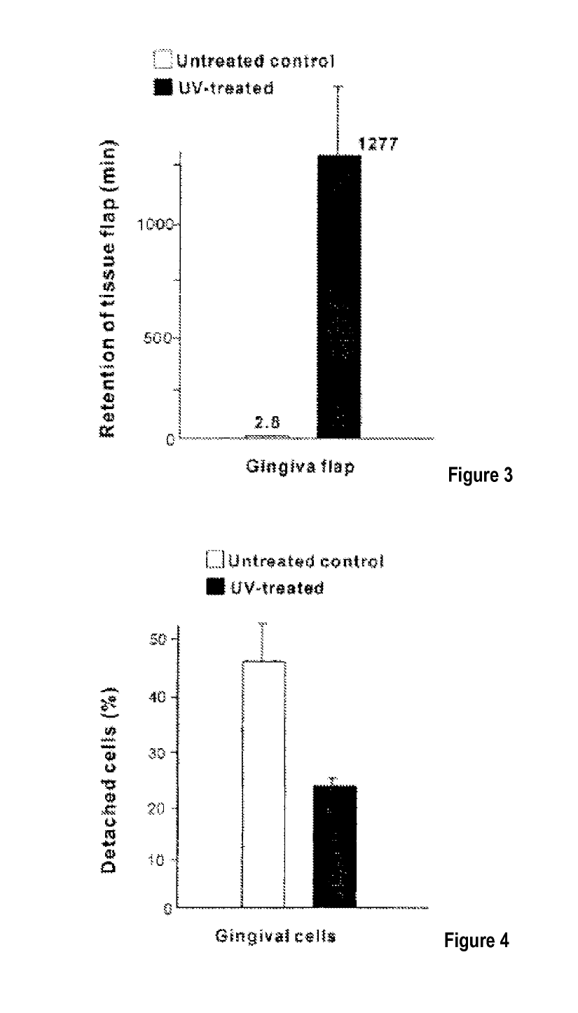

The 2 mm×2 mm gum tissues isolated from rat upper jaw were placed on gold alloy disks with and without UV treatment. The gum tissues were incubated in the culture medium for 2 days to obtain the initial attachment to titanium disks. Then, the culture dish was shaken on an agitating device to detach from titanium disks. The gum tissues were retained on UV treated titanium disks for over 1200 min without detachment, while the gum tissues on untreated titanium disks were detached within 3 min (FIG. 3).

### Enhanced Adhesion of Gum (Gingival) Cells on UV-Treated Metal

The gingival (epithelial) cells isolated from rat upper jaw were placed on titanium disks with and without UV treatment. The cells were incubated in the culture medium for 24 hours to obtain the initial attachment to titanium disks. Then, the culture dish was shaken on an agitating device for 25 min to detach from titanium disks. The number of detached cells was double on untreated titanium disks than on the UV-treated titanium disks (FIG. 4).

### Enhanced Adhesion of Fibroblasts Cells on UV-Treated Metal

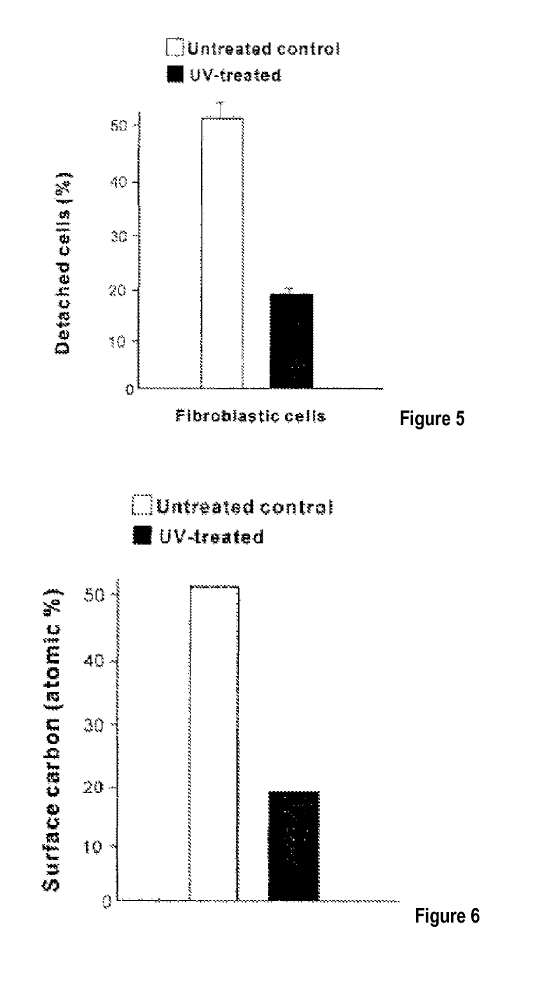

The NIH3T3 fibroblastic cells were placed on titanium disks with and without UV treatment. The cells were incubated in the culture medium for 24 hours to obtain the initial attachment to titanium disks. Then, the culture dish was shaken on an agitating device for 25 min to detach from titanium disks. The number of detached cells was 2.5 times greater on untreated titanium disks than on the UV-treated titanium disks (FIG. 5).

### Characteristics of UV-Treated Materials

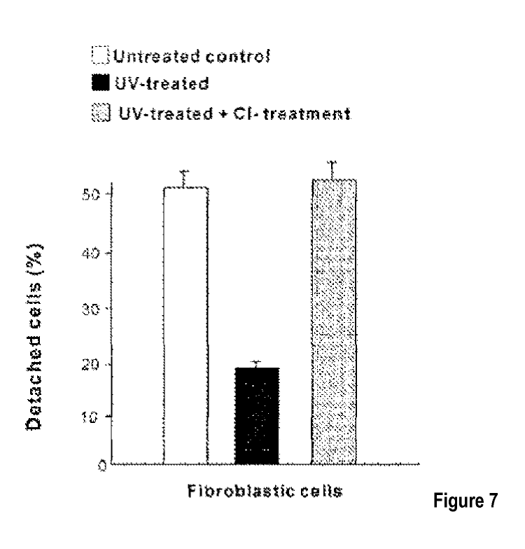

XPS measurement showed that UV-treated titanium surfaces showed a lower percentage of atomic carbon (smaller than 25%) than untreated titanium surfaces (above 45%) (FIG. 6). We also demonstrated the change of surface electric charge of UV treated metals. Because treating UV-treated titanium surfaces with monovalent anions, such as Cl—, abrogated the enhancement of cell adhesion, the UV-treated surfaces were found to be electro-positive (FIG. 7).

### Conclusion

The present studies show that UV light treatment of prosthetic materials significantly enhances the adhesion and retention of the soft tissues (gum and skin tissues) and soft-tissue cells, leading to a remarkably greater degree of soft tissue integration. Because the degree of soft tissue adhesion/integration determines the degree of soft tissue seal from the surrounding environments and protects the internal biological cells, tissues and structures, it can be an efficient and promising measure to maintain short- and long-term health of biological tissues around the prostheses and related devices.

### REFERENCES

[1] Schmidlin K, Schnell N, Steiner S, Salvi G E, Pjetursson B, Matuliene G, Zwahlen M, Bragger U, Lang N P. Complication and failure rates in patients treated for chronic periodontitis and restored with single crowns on teeth and/or implants. Clin Oral Implan Res 2010; 21:550.
[2] Huh Y H, Shin H J, Kim D G, Park C J, Cho L R. Full mouth fixed implant rehabilitation in a patient with generalized aggressive periodontitis. J Adv Prosthodont 2010; 2:154.
[3] Wennstrom J L, Ekestubbe A, Grondahl K, Karlsson S, Lindhe J. Oral rehabilitation with implant-supported fixed partial dentures in periodontitis-susceptible subjects. A 5-year prospective study. Journal of clinical periodontology 2004; 31:713.
[4] Lopez R, Dahlen G, Baelum V. Subgingival microbial consortia and the clinical features of periodontitis in adolescents. European journal of oral sciences 2011; 119:455.
[5] Rams T E, Flynn M J, Slots J. Subgingival microbial associations in severe human periodontitis. Clinical infectious diseases: an official publication of the Infectious Diseases Society of America 1997; 25 Suppl 2:S224.
[6] Geertman M E, Boerrigter E M, Van't Hof M A, Van Waas M A, van Oort R P, Boering G, Kalk W. Two-center clinical trial of implant-retained mandibular overdentures versus complete dentures-chewing ability. Community Dent Oral Epidemiol 1996; 24:79.
[7] van Kampen F M, van der Bilt A, Cune M S, Fontijn-Tekamp F A, Bosman F. Masticatory function with implant-supported overdentures. J Dent Res 2004; 83:708.
[8] Heydecke G, McFarland D H, Feine J S, Lund J P. Speech with maxillary implant prostheses: ratings of articulation. J Dent Res 2004; 83:236.
[9] Melas F, Marcenes W, Wright P S. Oral health impact on daily performance in patients with implant-stabilized overdentures and patients with conventional complete dentures. Int J Oral Maxillofac Implants 2001; 16:700.
[10] Nowjack-Raymer R E, Sheiham A. Association of edentulism and diet and nutrition in US adults. J Dent Res 2003; 82:123.
[11] Doundoulakis J H, Eckert S E, Lindquist C C, Jeffcoat M K. The implant-supported overdenture as an alternative to the complete mandibular denture. J Am Dent Assoc 2003; 134:1455.
[12] Annual industry report. US markets for dental implants: Executive summary. Implant Dent 2003; 12:108.
[13] Allegrini S, Jr., Allegrini M R, Yoshimoto M, Konig B, Jr., Mai R, Fanghanel J, Gedrange T. Soft tissue integration in the neck area of titanium implants—an animal trial. J Physiol Pharmacol 2008; 59 Suppl 5:117.
[14] Klinge B, Meyle J. Soft-tissue integration of implants. Consensus report of Working Group 2. Clin Oral Implan Res 2006; 17 Suppl 2:93.
[15] Touati B, Rompen E, Van Dooren E. A new concept for optimizing soft tissue integration. Pract Proced Aesthet Dent 2005; 17:711.
[16] Hermann J S, Buser D, Schenk R K, Higginbottom F L, Cochran D L. Biologic width around titanium implants. A physiologically formed and stable dimension over time. Clin Oral Implan Res 2000; 11:1.
[17] Cochran D L, Hermann J S, Schenk R K, Higginbottom F L, Buser D. Biologic width around titanium implants. A histometric analysis of the implanto-gingival junction around unloaded and loaded nonsubmerged implants in the canine mandible. Journal of periodontology 1997; 68:186.
[18] Dvorak G, Arnhart C, Heuberer S, Huber C D, Watzek G, Gruber R. Peri-implantitis and late implant failures in postmenopausal women: a cross-sectional study. Journal of clinical periodontology 2011; 38:950.
[19] Algraffee H, Borumandi F, Cascarini L. Peri-implantitis. The British journal of oral & maxillofacial surgery 2011.
[20] Bordin S, Flemmig T F, Verardi S. Role of fibroblast populations in peri-implantitis. The International journal of oral & maxillofacial implants 2009; 24:197.
[21] Carcuac 0, Jansson L. Peri-implantitis in a specialist clinic of periodontology. Clinical features and risk indicators. Swedish dental journal 2010; 34:53.
[22] Proussaefs P. Use of the frontal process of the maxillary bone for implant placement to retain a nasal prosthesis: a clinical report. The International journal of oral & maxillofacial implants 2004; 19:901.
[23] Zhang X, Chen S L, Zhang J M, Chen J L. Fabrication of a surgical template for orbital implant placement: a case report. The International journal of oral & maxillofacial implants 2010; 25:826.
[24] Gitto C A, Plata W G, Schaaf N G. Evaluation of the peri-implant epithelial tissue of percutaneous implant abutments supporting maxillofacial prostheses. The International journal of oral & maxillofacial implants 1994; 9:197.
[25] Hori N, Iwasa F, Ueno T, Takeuchi K, Tsukimura N, Yamada M, Hattori M, Yamamoto A, Ogawa T. Selective cell affinity of biomimetic micro-nano-hybrid structured TiO2 overcomes the biological dilemma of osteoblasts. Dental materials: official publication of the Academy of Dental Materials 2010; 26:275.
[26] Dalby M J, Gadegaard N, Curtis A S, Oreffo R O. Nanotopographical control of human osteoprogenitor differentiation. Curr Stem Cell Res Ther 2007; 2:129.
[27] Dalby M J, Gadegaard N, Wilkinson C D. The response of fibroblasts to hexagonal nanotopography fabricated by electron beam lithography. J Biomed Mater Res A 2008; 84:973.
[28] Smith L L, Niziolek P J, Haberstroh K M, Nauman E A, Webster T J. Decreased fibroblast and increased osteoblast adhesion on nanostructured NaOH-etched PLGA scaffolds. International journal of nanomedicine 2007; 2:383.
[29] Baxter L C, Frauchiger V, Textor M, ap Gwynn I, Richards R G. Fibroblast and osteoblast adhesion and morphology on calcium phosphate surfaces. Eur Cell Mater 2002; 4:1.
[30] Ogawa T, Nishimura I. Different bone integration profiles of turned and acid-etched implants associated with modulated expression of extracellular matrix genes. Int J Oral Maxillofac Implants 2003; 18:200.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A prosthetic device, having an enhanced soft tissue integration and seal, wherein the prosthetic device is treated by ultraviolet light (UV) for a period of time of sufficient length prior to implantation of the prosthetic device in a subject so as to impart electrostatic properties to the surface of the device, and wherein the enhanced soft tissue integration and seal is a soft tissue integration with and seal around the prosthetic device that is enhanced by about 10% or above as compared with a device without UV treatment.

2. The prosthetic device of claim 1, wherein the soft tissue comprises cells selected from gingival cells, epithelial cells, fibroblast cells, and combinations thereof.

3. (canceled)

4. The prosthetic device of claim 1, which is a dental implant or an orthopedic implant.

5. (canceled)

6. The prosthetic device of claim 4, wherein the dental implant is selected from the group consisting of dental crowns, bridges, implant fixtures, implant abutment components, attachments, bars, and a superstructure to retain and support prostheses that contact soft tissues, and wherein the orthopedic implant is selected from the group consisting of femoral stems, knee implants, spine screws, and plates.

7. (canceled)

8. The prosthetic device of claim 1, comprising gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

9. The prosthetic device of claim 1, selected from the group consisting of jaw bone prosthetic device, repairing and stabilizing screws, pins, frames, and plates for bone, spinal prosthetic devices, femoral prosthetic devices, neck prosthetic devices, knee prosthetic devices, wrist prosthetic devices, joint prosthetic devices, maxillofacial prosthetic, limb prostheses for conditions resulting from injury and disease, and combinations thereof.

10. The prosthetic device of claim 1, comprising a polymeric material or a bone cement material.

11. The prosthetic device of claim 10, wherein the bone cement material comprises a material selected from the group consisting of polyacrylates, polyesters, poly(methyl methacrylate) (PMMA) or methyl methacrylate (MMA), bioglass, ceramics, calcium-based materials, calcium phosphate-based materials, and combinations thereof.

12. The prosthetic device of claim 1, wherein the UV light has an intensity of about 0.05 mW/cm² to about 4.0 mW/cm² of a wave length from about 400 nm to about 100 nm.

13. The prosthetic device of claim 1, wherein the electrostatic properties comprise positive charges ranging from 0.01 nC to 10.00 nC.

14. A method, comprising treating a prosthetic device with ultraviolet light (UV) for a period of time of sufficient length prior to implantation of the prosthetic device in a subject so as to impart electrostatic properties to the surface of the device, and wherein the enhanced soft tissue integration and seal is a soft tissue integration with and seal around the prosthetic device that is enhanced by about 10% or above as compared with a device without UV treatment.

15. The method of claim 14, wherein the period of time is about 20 minutes or longer.

16. The method of claim 14, wherein the UV light is has an intensity of about 0.05 mW/cm² to about 4.0 mW/cm² of a wave length from about 400 nm to about 100 nm.

17. The method of claim 14, wherein the electrostatic properties comprise positive charges ranging from 0.01 nC to 10.00 nC.

18. The method of claim 14, wherein the prosthetic device comprises a metallic material.

19. The method of claim 14, wherein the prosthetic device comprises gold, platinum, tantalum, niobium, nickel, iron, chromium, titanium, titanium alloy, titanium oxide, cobalt, zirconium, zirconium oxide, manganese, magnesium, aluminum, palladium, an alloy formed thereof, or combinations thereof.

20. The method of claim 14, wherein the prosthetic device is selected from the group consisting of tooth prosthetic devices, jaw bone prosthetic device, repairing and stabilizing screws, pins, frames, and plates for bone, spinal prosthetic devices, femoral prosthetic devices, neck prosthetic devices, knee prosthetic devices, wrist prosthetic devices, joint prosthetic devices, maxillofacial prosthetic, limb prostheses for conditions resulting from injury and disease, and combinations thereof.

21. The method of claim 14, wherein the prosthetic device comprises a polymeric material or a bone cement material.

22. The method of claim 21, wherein the bone cement material comprises a material selected from the group consisting of polyacrylates, polyesters, poly(methyl methacrylate) (PMMA) or methyl methacrylate (MMA), bioglass, ceramics, calcium-based materials, calcium phosphate-based materials, and combinations thereof.

23. A method, comprising implanting a prosthetic device in a subject in need thereof, wherein the prosthetic device is according to claim 1.
